(19) United States
(12) Patent Application Publication
(10) Pub. No.: US 2026/0055384 A1
(43) Pub. Date: Feb. 26, 2026

(54) METHOD FOR OBTAINING A LYOPHILISED ENZYMATIC PREPARATION AND ITS USES IN SYNTHESIS REACTIONS FOR THE PRODUCTION OF ESTOLIDE-TYPE BIOLUBRICANTS, BIODIESEL AND POLYOL-ESTERS-TYPE BIOLUBRICANTS

(71) Applicants: Petróleo Brasileiro S.A. – Petrobras, Rio de Janeiro (BR); Universidade Federal do Rio de Janeiro — UFRJ, Rio de Janeiro (BR)

(72) Inventors: Denise Maria Guimarães FREIRE, Rio de Janeiro (BR); Elisa D’avila Costa CAVALCANTI, Rio de Janeiro (BR); Charles Lima Bessa ASSUNCAO, Rio de Janeiro (BR); Ana Cristina DE ARAUJO COLLAÇO, Rio de Janeiro (BR); Jose Andre Cavalcanti DA SILVA, Rio de Janeiro (BR); Erika Cristina Gonçalves AGUIEIRAS, Duque de Caxias (BR)

(21) Appl. No.: 19/273,862

(22) Filed: Jul. 18, 2025

(30) Foreign Application Priority Data
Aug. 26, 2024 (BR) 1020240174771

(51) Int. Cl. C12N 9/20; C12P 7/6436; C12P 7/649

(52) U.S. Cl. CPC C12N 9/20; C12P 7/6436; C12P 7/649; C12Y 301/01003

(57) ABSTRACT

The present disclosure describes a method for obtaining a proprietary biocatalyst, called lyophilized enzyme preparation (PEL) via submerged fermentation (SmF) of Diutina rugosa yeast. The biocatalyst obtained by the process is also applied in synthesis reactions for the production of estolide-type biolubricants; biodiesel and polyol-esters-type biolubricants.

### CROSS-REFERENCE TO RELATED APPLICATIONS

This application claims priority to Brazilian Patent Application No. 1020240174771, filed Aug. 26, 2024, which is incorporated herein in its entirety by reference thereto.

### FIELD

The present disclosure has as its field of application industrial plants for the production of biodiesel and biolubricants from vegetable oils. At the same time, the present disclosure falls within the field of production of enzymatic biocatalysts that are used in synthesis reactions to obtain biodiesel and biolubricants from vegetable oils.

### BACKGROUND

Biolubricants derived from vegetable oils are considered an alternative to the use of petroleum-derived lubricants, since they are biodegradable organic esters, that is, they can be decomposed through microbial metabolism, in addition to presenting physicochemical properties similar to lubricants of mineral origin.

In the conventional process of production of biolubricants, acidic chemical catalysts, such as sulfuric acid and perchloric acid, are used at high temperatures (200° C.) and pressures for the esterification reaction of fatty acids. However, in addition to generating acidic effluents, the use of strong acids can lead to the breakdown of functional groups, releasing sulfonated acid groups that reduce the pH, compromising process performance.

Another product of industrial interest is biodiesel. Biodiesel is produced industrially from a transesterification reaction, in which methanol reacts with the triglyceride present in vegetable oil through the action of a homogeneous catalyst (usually NaOH and KOH), generating a mixture of fatty acid methyl esters (biodiesel) and one mole of glycerin for each mole of triglyceride. Although the use of homogeneous alkaline catalysts has high reaction rates, being an already consolidated and low-cost process, the use of these catalysts has many disadvantages, such as the difficulty in separating them from the product, the possibility of saponification reactions when using raw material with a high content of free fatty acids (>0.5% by mass), high energy consumption and large amounts of alkaline wastewater. Thus, enzymatic synthesis is shown to be an interesting alternative for obtaining biolubricants and biodiesel in relation to conventional chemical methods.

Since enzymes are catalysts with high specificity, low concentrations of reaction byproducts are generated, which reduces the amount of industrial effluents. In addition, the energy consumption of the process is reduced, because the enzymes act in mild reaction conditions of temperature, pH and pressure. Considering enzymatic synthesis, the use of proprietary lipases produced by submerged fermentation as proposed by the present disclosure appears to be an even more promising alternative when compared to the use of high-cost commercial lipases and the use of chemical catalysts.

Commercial lipase from Diutina rugosa (Lipomod 34 MDP) has been used in biodiesel production reactions, showing good results in terms of conversion to esters.

As examples, MENDEZ et al., (2022), evaluated the production of biodiesel via transesterification of coconut oil, obtaining a biodiesel yield of 98%. In addition, IULIANO et al., (2020), used lipase from Diutina rugosa for the conversion of beer pomace oil (BSGs) into biodiesel, in the presence of methanol, with a yield of 98%. XIE et al., (2020), evaluated an organic copolymer to be encapsulated in Fe₃O₄ nanoparticles in which lipase from Diutina rugosa was bound. The biodiesel yield was maintained at 79.4% after reuse for five cycles. Furthermore, this lipase has been used to obtain biolubricants from polyols such as trimethylolpropane (TMP), neopentyl glycol (NPG) and pentaerythritol (PET) as substrates. PAPADAKI et al., (2018), used microbial oil from Rhodosporidium toruloides and Cryptococcus curvatus to produce biolubricants with NPG. Conversions higher than 82% were achieved. On the other hand, the palm oil deodorizer distillate (POD) consists of another substrate that can be used with NPG or TMP, as described by FERNANDES et al., (2018), reaching maximum esterification of OH to POD-TMP esters (94%) and POD-NPG esters (87%) using 4% Lipomod 34 MDP. FERNANDES et al., (2021) evaluated the synthesis of biolubricants from distilled soybean fatty acids (DDOS) using Diutina rugosa lipase in free and immobilized form using NPG and TMP as polyalcohols. In addition, recent studies report the use of this enzyme in estolide synthesis reactions (biolubricants) using castor bean free fatty acid (LMFA) as substrate. GRECO-DUARTE et al., (2017) described that Lipomod 34 MDP has greater specificity for ricinoleic acid compared to polyols. The reduction in acidity in the reaction with LMFA was similar to that obtained in the presence of polyols. Two other commercial enzymes, from Rhizomucor miehei and Candida antarctica, were not able to reduce acidity in this reaction medium without polyols. GRECO-DUARTE et al (2019) also described a kinetic means of controlling the size of estolides, in which the analysis showed that estolides of different sizes [dimers, trimers, tetramers and pentamers (+)] were produced throughout the reaction time.

Within the patent publications, the patent BR 102016020837-8 filed on Sep. 9, 2016, describes an integrated process for producing biodiesel from the acid oil of the pulp of the macauba fruit (Acrocomia aculeata) through the transesterification route catalyzed by biocatalysts, using the macauba cake as a solid-state fermentation culture medium to obtain lipases.

The patent application BR 102017022583-6 filed on Oct. 20, 2017, describes a process for the production of esters and biolubricants using solid enzymatic preparations produced by solid-state fermentation with a methodology very similar to that described in the patent number BR 102016020837-8. Although this document has points in common with the disclosure under study, the disclosure being described addresses a process for the production and application of a proprietary biocatalyst, called lyophilized enzymatic preparation (PEL), produced by submerged fermentation using the Diutina rugosa yeast. These activities differ from the patent application BR 102017022583-6, which addresses the production and application of a biocatalyst produced by solid-state fermentation using the filamentous fungus Rhizomucor miehei. The Diutina rugosa yeast is also mentioned in the document, but it is not obtained by a submerged fermentation process and is not used in its raw form, which is a disadvantage compared to the present disclosure. Therefore, the present disclosure deals with a promising process at the national level, since Brazil is rich in a diversity of raw materials for the production of biolubricant and biodiesel, having the potential to contribute to the biodiesel and biolubricant market, as it uses its own specific biocatalyst produced by submerged fermentation.

Furthermore, since the present disclosure involves obtaining biodiesel and biolubricants from renewable sources, it contributes to the decarbonization of the energy matrix and, consequently, the mitigation of climate change, in addition to promoting the cultivation of plants in new areas, increasing income and technological advances in the agribusiness sector.

### SUMMARY

The present disclosure describes a method for obtaining a proprietary biocatalyst, called lyophilized enzyme preparation (PEL) via submerged fermentation of Diutina rugosa yeast. The biocatalyst obtained by the process is also applied in synthesis reactions for the production of biodiesel and biolubricants. This enzyme is capable of catalyzing biodiesel and biolubricant synthesis reactions with superior performance to the commercial enzyme and under milder process conditions, which constitutes a major advance in the oleochemical sector.

### BRIEF DESCRIPTION OF THE FIGURES

FIG. 1 is a block diagram showing the production of the lyophilized enzyme preparation (biocatalyst).

FIGS. 2A and 2B are block diagrams of the synthesis reactions for the production of estolide-type biolubricants.

FIG. 3 is a block diagram of the synthesis reactions for the production of biodiesel.

FIGS. 4A and 4B are block diagrams of the synthesis reactions for the production of polyol-ester-type biolubricants.

FIG. 5 shows the results obtained by the SDS-PAGE technique under denaturing conditions of Lipomod 34 MDP and PEL obtained from extraction with sodium phosphate buffer pH 7.0 (0.05 M), where A: Molecular mass standard; B: Lipomod 34 MDP; and C: PEL.

FIG. 6 shows the results obtained by the SDS-PAGE technique under partially denaturing conditions (A, B and C) and the zymogram (D and F) of Lipomod 34 MDP and PEL, where A: Molecular mass standard; B: Lipomod 34 MDP; C: PEL; D: Lipomod 34 MDP; and F: PEL.

FIG. 7 is a graph of the conversion obtained in the use and reuse reactions of the lyophilized enzyme preparation (PEL) in synthesis reactions of estolide-type biolubricants. The reactions were conducted with AGLM and 300 U of lyophilized enzyme preparation, under stirring at 200 rpm and at a temperature of 35 to 45° C. under atmospheric pressure.

FIG. 8 is a graph of the acidity and amount of free ricinoleic acid of the estolide synthesis reactions performed to characterize the physical and functional properties. The reactions were conducted with AGLM and 20% (w/w) of lyophilized enzyme preparation, under stirring at 200 rpm and a temperature of 35 to 45° C. under atmospheric pressure.

### DETAILED DESCRIPTION

The present disclosure describes a method for obtaining a biocatalyst called lyophilized enzyme preparation (PEL) obtained by submerged fermentation (SF) and its application in biodiesel and biolubricant synthesis reactions, which constitutes a major advance in the oleochemical sector.

### Production of PEL (Biocatalyst)

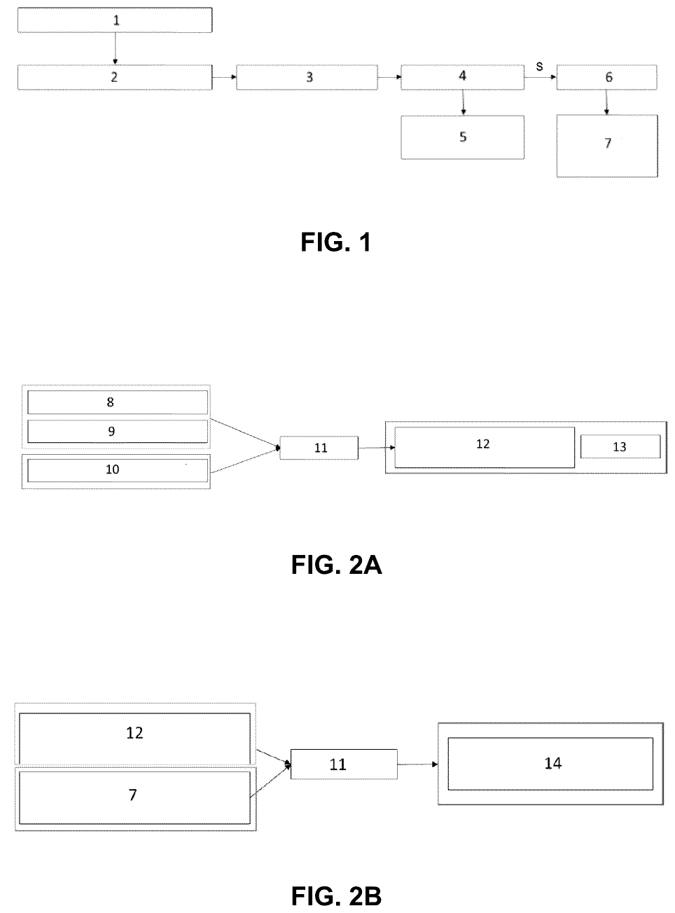

The Diutina rugosa yeast (1) is used for the production of lipases by submerged fermentation (2) using a fed-batch scheme of a carbon source (oleic acid) at a feed rate of 0.43 to 2 g/l/h and with bioprocess variables stipulated in the following ranges: temperature of 30 to 40° C., aeration of 0.5 to 3 vvm, pH of 6 to 7, agitation of 200 to 600 rpm. At the end of fermentation, the fermented medium (3) is subjected only to a centrifugation process (4), generating cells (5) and the supernatant (S) that is subjected to a lyophilization process (6) obtaining the lyophilized enzymatic preparation (7), capable of being used in synthesis reactions, as shown in FIG. 1. The PEL (biocatalyst) does not need to be purified, being used in its raw form in synthesis reactions.

### Synthesis Reactions for the Production of Estolide-Type Biolubricants

For estolide synthesis reactions, hydrolysis of castor oil is necessary to obtain castor free fatty acid (LMFA) (12). For this purpose, castor oil (8) and acetate buffer (100 mM, pH 4) (9) are used as substrates and castor seed (10) is used as biocatalyst in a seed/buffer/oil ratio of 1:5:5 (w/v/v). The reaction is carried out in a reactor (11) under stirring at 200 rpm and a temperature of 30 to 40° C., under atmospheric pressure. After 24 h, castor free fatty acid (12) and glycerol (13) are obtained as products. Castor free fatty acid (12) is recovered by extraction with ethyl acetate and glycerol (13) can be recovered and used in other processes. The castor oil-free fatty acid (12) obtained is used as a substrate in estolide synthesis reactions using the lyophilized enzyme preparation (7) as a biocatalyst under stirring at 200 rpm and a temperature of 35 to 45° C., under atmospheric pressure in a reactor (11). After 48 h, the estolides (14) are obtained as lubricant products, as shown in FIGS. 2A and 2B.

### Synthesis Reactions for Biodiesel Production

For biodiesel synthesis reactions, the distillate from the deodorization of palm oil (POD) (15) and hydrated ethanol (16) are used as substrates and the lyophilized enzyme preparation (7) is used as a biocatalyst under stirring at 200 rpm and a temperature of 40 to 50° C., under atmospheric pressure. The reactions are carried out in the molar ratio hydrated ethanol (95%):POD of 1:1 in reactor (11) and after 48 h, ethyl esters (17) and water (18) are obtained as reaction products.

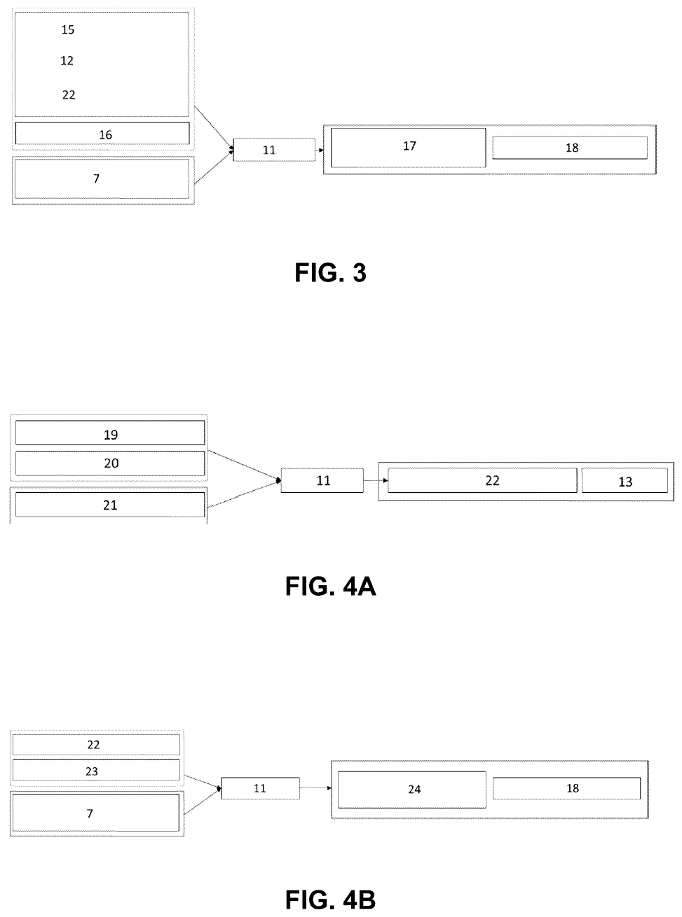

In this scheme, in addition to POD (15), it is possible to use both castor oil free fatty acid (FLFA) (12) and soybean free fatty acid (SLFA) (22) as substrates for the biodiesel synthesis reaction, as shown in FIG. 3.

### Synthesis Reactions for the Production of Polyol-Ester Biolubricants

For the synthesis reactions of polyol-ester biolubricants, the hydrolysis of soybean oil is necessary to obtain soybean free fatty acid (SLFA) (22). Thus, the hydrolysis occurs using soybean oil (19) and sodium phosphate buffer (100 mM; pH 7.0) (20) as substrates and the commercial enzyme Diutina rugosa (Lipomod 34 MDP) (21) as a biocatalyst. The reaction is carried out using a 1:1 (v/v) soybean oil/phosphate buffer ratio and Lipomod 34MDP (1% m/v), under stirring at 200 rpm and a temperature of 30 to 40° C., under atmospheric pressure in a reactor (11). After 4 h, soybean free fatty acid (SFFA) (22) and glycerol (13) are obtained as products. SFFA (22) is recovered by extraction with hexane and glycerol (13) can be recovered and used in other processes. For the synthesis reactions of polyol-ester biolubricants, SFFA (22) obtained from the hydrolysis of soybean oil and polyalcohol (Neopentylglycol—NPG) (23) are used as substrates in a NPG:SFFA molar ratio of 1:2.5. For this, the lyophilized enzyme preparation (7) is used as a biocatalyst. The reaction is carried out in a reactor (11) under stirring at 200 rpm and a temperature of 35 to 45° C., under atmospheric pressure, as shown in FIGS. 4A and 4B. After 48 h, the polyol esters (24) are obtained. In addition to the polyol esters, water (18) is generated as a co-product of the reaction. In the present disclosure, NPG was used as the polyalcohol. However, it is possible to use other polyalcohols such as trimethylolpropane (TMP) and pentaerythritol (PET).

### Test Results/Comparative Data

### Protein Profile

The lyophilized enzyme preparation (PEL) was produced from submerged fermentation (SF), according to the steps in the block diagram of FIG. 1, and the protein profile was characterized by the SDS-PAGE and zymography techniques. In addition to the evaluation of PEL, the commercial enzyme from Diutina rugosa (Lipomod 34 MDP) was also characterized by SDS-PAGE and zymography in order to compare the two products.

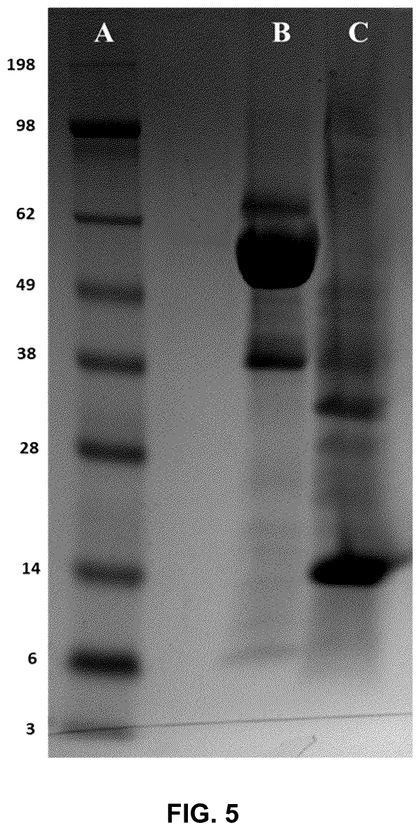

From the results of this technique, it was observed that the commercial lipase from Diutina rugosa (Lipomod 34 MDP) and the PEL produced in the present disclosure do not correspond to the same product. These differences are evidenced from the protein profile of both biocatalysts (FIG. 5). Two protein bands of approximately 30 to 40 kDa and a protein band of 14 kDa were observed in the lyophilized enzyme preparation produced in the present disclosure (image C). On the other hand, in relation to the commercial enzyme Lipomod 34 MDP, a majority band of 60 kDa and a band of 40 kDa were observed (image B). Image A shows the molecular mass pattern.

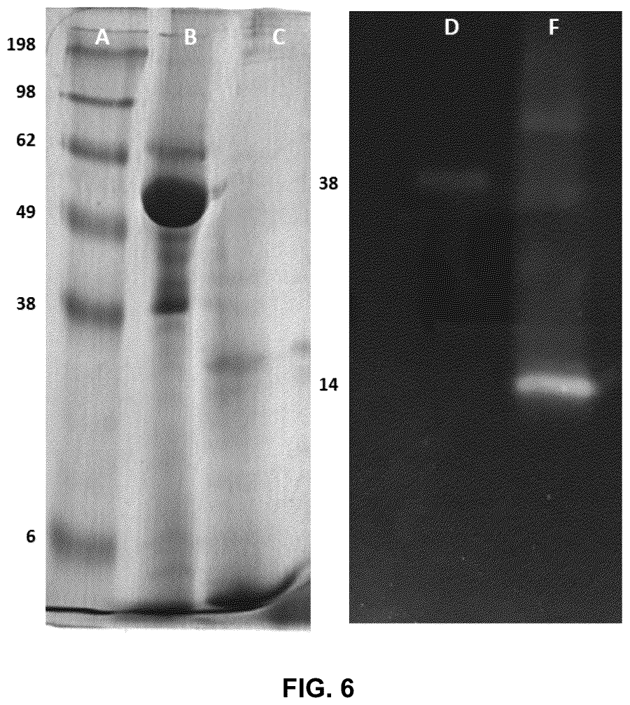

The PEL zymogram showed that the enzyme capable of catalyzing the synthesis of estolides possibly has a molecular mass of less than 40 kDa, unlike what was observed for Lipomod 34 MDP (FIG. 6). In addition to not representing the same product, the method for obtaining the commercial enzyme Lipomod 34 MDP may involve purification and formulation steps, unlike obtaining PEL, which is used in its raw form without the need for purification and formulation. Therefore, there is a very promising potential for using PEL as a biocatalyst in synthesis reactions.

### Standardization of Enzymatic Activity

In order to establish a better comparison between the applications of PEL and the commercial enzyme Lipomod 34 MDP, an enzymatic activity standardization was performed, in which each synthesis reaction contained a hydrolytic activity of 300 U. From the conversion values, it was possible to conclude that the lyophilized enzymatic preparation (PEL) showed a better performance when compared to the use of Lipomod 34 MDP in the synthesis reactions of biodiesel and biolubricants, evidencing the potential of the enzyme produced in the present disclosure (Table 1).

TABLE 1
Conversion results in the synthesis reactions of biodiesel
and biolubricants using the lyophilized enzymatic preparation
(300 U) or Lipomod 34 MDP (300 U or 4,000 U).

| | | Lipomod 34 | Lipomod 34 |
|---|---|---|---|
| | PEL | MDP | MDP |
| | (300 U) | (300 U) | (4,000 U) |
| Biodiesel | 80.2 ± 1.7% | 2.1 ± 1.1% | 59.17 ± 15.7% |
| Polyol-ester type | 83.3 ± 0.3% | 65.5 ± 0.3% | 82.4 ± 0.51% |
| biolubricant | | | |
| Estolide type | 90.3 ± 1.0% | 3.8 ± 1.2% | 56.3 ± 5.98% |
| biolubricant | | | |
| Biocatalyst mass | 1.23 g (30%) | 0.012 g (0.3%) | 0.16 g (4%) |

Table 1 addresses the conversions in the synthesis reactions of biodiesel and biolubricants using PEL (300 U of enzymatic activity) and Lipomod 34 MDP (300 U or 4,000 U of enzymatic activity). The results show that even using an enzymatic activity approximately 13 times greater for the commercial enzyme Lipomod 34 MDP (4,000 U), better conversion results were achieved with the lyophilized enzymatic preparation (300 U). This result shows the potential use of the lyophilized enzymatic preparation to replace the high-cost commercial enzymes on the market.

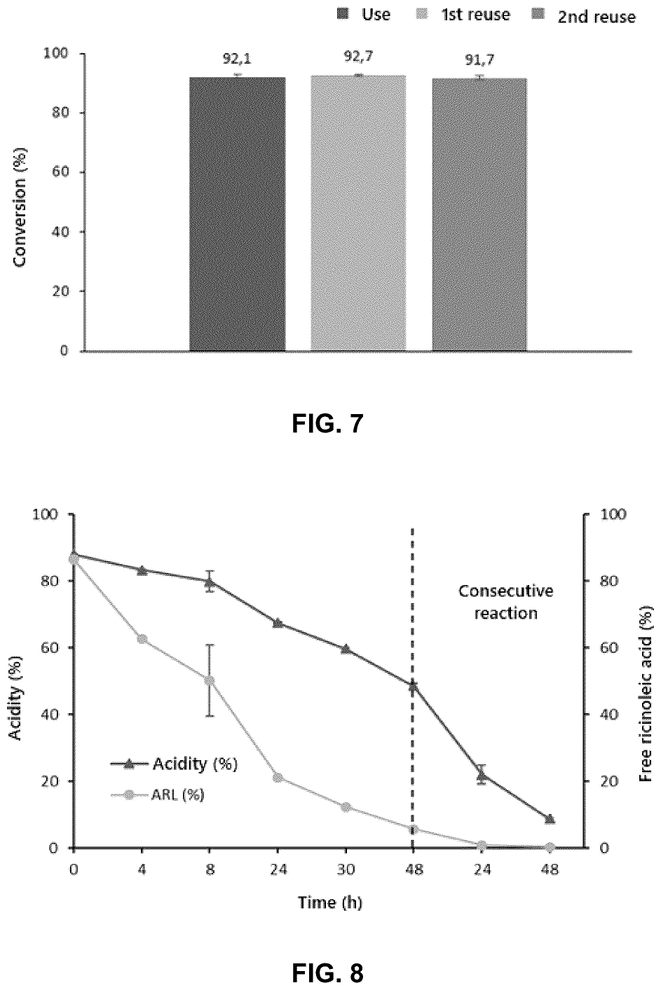

Due to the excellent results in the reactions for producing estolide-type biolubricants, the potential for reuse in these synthesis reactions was evaluated, and it was possible to reuse the enzyme at least twice, without loss of enzymatic activity and reduction in conversion, as shown in FIG. 7.

As previously described, to obtain PEL, the cell-free culture medium obtained from submerged fermentation was lyophilized without any purification and/or formulation steps. In addition to presenting an unprecedented electrophoretic profile (different enzymes were obtained by this methodology as observed by SDS-PAGE and zymogram), PEL can be reused at least twice in estolide synthesis reactions and this result may be associated with the high stability and specificity of this biocatalyst. It is worth mentioning that the possibility of reusing the enzyme favors the technical-economic viability of the technology. Table 2 shows the main differences in enzyme reuse in estolide synthesis reactions, both of the commercial enzyme Lipomod 34 MDP (results obtained in the work of Greco-Duarte 2018) and of the enzyme used in the present disclosure (PEL).

TABLE 2
Comparison of the reuse potential of the commercial
enzyme Lipomod 34 MDP and the PEL produced by SmF
in estolide-type biolubricant synthesis reactions.

| | Commercial | |
|---|---|---|
| | enzyme Lipomod | PEL produced |
| Enzyme | 34 MDP * | by SmF |
| Amount of enzyme reuses | 4 | 2 |
| Duration of each enzymatic reaction | 5 h | 48 h |
| Total time of enzyme use | 25 h | 144 h |
| Loss of enzyme activity after use | Yes | No |

* Results from Greco-Duarte, 2018.

### Estolide Characterization

For estolide characterization, the reaction medium was composed of PEL and AGLM. After 48 h, the reaction medium (product and lyophilized enzyme preparation) was centrifuged to separate the phases. After this procedure, the product was subjected to a consecutive reaction of 48 h, using the recovered lyophilized enzyme preparation. Aliquots from times 0, 4, 8, 24, 30, and 48 hours and from times 24 h and 48 h of the consecutive reaction were evaluated for the free ricinoleic acid content (FIG. 8). The final product from the consecutive reaction showed a final acidity of 8.79±0.275% and a free ricinoleic acid concentration of 0.37±0.007, as shown in FIG. 8.

The results of the characterization of the final product are reported in Table 3 together with characterization data of estolides produced using AGLM and the commercial enzyme Lipomod 34 MDP described by GRECO-DUARTE (2018) under non-optimized and optimized reaction conditions. Analyses of viscosity measured at 40° C. (mm²/S), viscosity measured at 100° C. (mm²/s), viscosity index, oxidative stability (min), pour point (° C.) and total acidity index (mg KOH/g of sample) were performed.

TABLE 3
Characterization regarding the lubricating characteristics of the estolides obtained
in the reaction conducted at 35 to 45° C. containing 20% (w/w) of lyophilized
enzyme preparation. The reaction in the non-optimized condition in the work of
GRECO-DUARTE (2018) was carried out at 40° C., 4% (w/w) of Lipomod 34 MDP
and 15% (w/w) of water, while the reaction in the optimized condition was carried
out at 45° C., 1.61% (w/w) of Lipomod 34 MDP and 14.4% (w/w) of water.

| | | | | | | TAI* |
|---|---|---|---|---|---|---|
| | V | V | | OS* | PP* | (mg KOH/g |
| | 40° C.* | 100° C.* | VI* | (min) | (° C.) | of sample) |
| Present disclosure | 451.8 | 49.92 | 172 | 43 | −42 | 28 |
| GRECO-DUARTE (2018) - | 153.7 | 19.20 | 142 | 37 | −45 | 57.6 |
| non-optimized condition | | | | | | |
| GRECO-DUARTE (2018) - | 275.5 | 29.11 | 142 | 48 | −42 | 51.58 |
| optimized condition | | | | | | |

*V 40° C.—Average viscosity at 40° C. (mm²/s);
V 100° C.—Average viscosity at 100° C. (mm²/s)
VI—viscosity index;
OS—oxidative stability (min);
PP—pour point (° C.);
TAI—total acidity index (mg KOH/g sample)

As observed in Table 3, the estolide produced in the present disclosure has very promising physical and functional properties when compared to that obtained using the commercial enzyme Lipomod 34 MDP. The product obtained can be used as the main component of a lubricating oil (base oil), being classified within group V according to the American Petroleum Institute (API). Thus, the results obtained in the present disclosure indicate the achievement of a new enzyme from Diutina rugosa produced by submerged fermentation, capable of catalyzing reactions of biodiesel and biolubricant synthesis with a performance superior to the commercial enzyme. Furthermore, the use of this enzyme is capable of generating a biolubricant with promising characteristics when compared to the biolubricant produced by the commercial enzyme Lipomod 34 MDP.

1. A method for obtaining a lyophilized enzyme preparation (PEL), the method comprising the steps of:
obtaining a fermented medium by submerged fermentation (SmF) of Diutina rugosa yeast;
centrifuging the fermented medium, after completion of fermentation, to obtain cells and a supernatant; and
lyophilizing the supernatant to obtain PEL as a biocatalyst.

2. The method according to claim 1, wherein the submerged fermentation is in a batch fed with oleic acid as a carbon source at a feed rate of 0.43 to 2 g/l/h.

3. The method according to claim 1, wherein the submerged fermentation occurs at: a temperature of 30° C. to 40° C.; an aeration of 0.5 to 3 vvm; a pH of 6 to 7; and an agitation of 200 to 600 rpm.

4. The method according to claim 1, wherein the PEL is not purified and is not formulated.

5. The method according to claim 1, wherein the PEL is in its crude form, and
wherein the method further comprises synthesizing the PEL to produce at least one of an estolide-type biolubricants; a biodiesel; or polyol-ester type biolubricants.

6. The method according to claim 5, wherein the synthesizing comprises production of the estolide-type biolubricants, wherein castor oil-free fatty acid is used as a substrate, and wherein the PEL is used as a biocatalyst.

7. The method according to claim 6, wherein the synthesizing occurs in a reactor under stirring at 200 rpm, at a temperature of 35° C. to 45° C., and under atmospheric pressure.

8. The method according to claim 5, wherein the synthesizing comprises the production of the biodiesel, wherein palm oil deodorizer distillate (POD) and hydrated ethanol (95%) are used as substrates or castor oil free fatty acid or soybean free fatty acid as substrates, and wherein the PEL is used as a biocatalyst.

9. The method according to claim 8, wherein the synthesizing occurs in a reactor under stirring at 200 rpm, at a temperature of 40° C. to 50° C., under atmospheric pressure, and in a molar ratio of hydrated ethanol (95%):POD of 1:1.

10. The method according to claim 5, wherein the synthesizing comprises the production of polyol-ester type biolubricants, wherein soybean free fatty acid and polyalcohol are used as a substrate, and wherein PEL is used as a biocatalyst.

11. The method according to claim 10, wherein the synthesizing occurs in a reactor under stirring at 200 rpm, at a temperature of 35° C. to 45° C., and under atmospheric pressure, and wherein a molar ratio of polyalcohol to soybean free fatty acid is.

12. The method according to claim 10, wherein the polyalcohol is trimethylolpropane (TMP) or pentaerythritol (PET).
